(19) United States
(12) Patent Application Publication
(10) Pub. No.: US 2026/0061167 A1
(43) Pub. Date: Mar. 5, 2026

(54) VASCULAR PUNCTURE SHEATH FACILITATING FIXATION AND REDIRECTION AND VASCULAR PUNCTURE DEVICE

(71) Applicant: Yin Xia, Fuzhou (CN)

(72) Inventor: Yin Xia, Fuzhou (CN)

(21) Appl. No.: 19/366,303

(22) Filed: Oct. 22, 2025

(30) Foreign Application Priority Data
Aug. 29, 2024 (CN) 202422117256.2

(51) Int. Cl. A61M 25/06; A61M 25/00; A61M 25/04

(52) U.S. Cl. CPC A61M 25/0662; A61M 25/0068; A61M 25/0074; A61M 25/04; A61M 2025/0004; A61M 2025/0079; A61M 2205/0216; A61M 2205/0238; A61M 2205/0266; A61M 2205/586; A61M 2210/12

(57) ABSTRACT

A vascular puncture sheath facilitating fixation and redirection comprises: a puncture base; an inner sheath tube, connected to lower end of the puncture base; an outer sheath tube, movably sleeved on outside of the inner sheath tube; a metal stent, fixedly arranged at lower end of the outer sheath tube and used for fixing the outer sheath tube in the blood vessel; wherein, a chamber is provided between the inner sheath tube and the outer sheath tube; a tube wall of the outer sheath tube is provided with a plurality of through-holes communicating with the chamber, the metal stent includes a plurality of memory metal wires, lower ends of the memory metal wires are fixed on outer wall of the outer sheath tube, and upper ends of the memory metal wires pass through corresponding through-holes into the chamber and are connected to tube wall of the inner sheath tube.

### TECHNICAL FIELD

The invention relates to the technical field of medical devices, and specifically to a vascular puncture sheath facilitating fixation and redirection.

### BACKGROUND ART

A vascular puncture sheath is a medical device used for vascular puncture. Its main function is to establish a surgical passage from the skin to the blood vessel, allowing surgical instruments such as catheters and guidewires to enter the blood vessel and reach the lesion site.

Currently, common vascular puncture sheaths have many problems during surgical procedures. For example, the sheath tube is difficult to maintain stable puncture in the blood vessel during surgery, and the sheath tube often falls off during the operation. This requires medical staff to perform a second puncture on the patient, increasing the difficulty of the operation and the harm to the patient. In addition, when it is necessary to treat blood vessels in different directions or parts very close to the same end of a blood vessel and it is difficult to access the target blood vessel, or when surgery needs to be performed on the other end of the blood vessel, existing vascular sheaths cannot adjust their direction; in such cases, a second puncture is always needed to align the vascular puncture sheath with the target blood vessel. This second puncture not only increases surgical difficulty but also raises the risk of puncture-related complications and inflicts more harm on the patient. Moreover, anatomical specificity and complexity often lead to puncture failure, which can affect the overall outcome of the surgery.

Secondly, due to the poor stability of the sheath tube in the blood vessel, when the clinician inserts the guidewire into the sheath tube and swings the guidewire, the guidewire will inevitably come into contact with the sheath tube, thereby driving the sheath tube to shift. This phenomenon further increases the difficulty for clinicians to operate the guidewire for redirection, making the surgical process take longer and requiring higher surgical experience from clinicians.

### SUMMARY OF THE INVENTION

The purpose of the invention is to provide a vascular puncture sheath facilitating fixation and redirection, which can prevent the puncture sheath from detaching from the blood vessel during surgery, thereby improving the stability of the puncture sheath in the blood vessel and further assisting clinicians in operating the guidewire for redirection, and making it easier to access the target blood vessel.

The purpose of the invention is achieved through the following technical solutions:
a vascular puncture sheath facilitating fixation and redirection, comprising:
a puncture base;
an inner sheath tube, connected to a lower end of the puncture base;
an outer sheath tube, movably sleeved on an outside of the inner sheath tube;
a metal stent, fixedly arranged at a lower end of the outer sheath tube and used for fixing the outer sheath tube in the blood vessel.

Wherein, a longitudinally extending chamber is provided between the inner sheath tube and the outer sheath tube; a tube wall of the outer sheath tube is provided with a plurality of through-holes communicating with the chamber, the metal stent includes a plurality of memory metal wires, lower ends of the memory metal wires are fixed on an outer wall of the outer sheath tube, and upper ends of the memory metal wires pass through the corresponding through-holes into the chamber and are connected to a tube wall of the inner sheath tube;
an adjustment mechanism located above the metal stent is also provided in the chamber, the outer sheath tube moves relative to the inner sheath tube, thereby driving the adjustment mechanism to control the expansion or contraction of the metal stent.

Compared with the prior art, the advantages of the invention are as follows:
1. Through the relative sliding between the outer sheath tube and the inner sheath tube and the cooperation of the adjustment mechanism located in the chamber, the metal stent can expand or contract in the blood vessel according to the surgical needs. Thus, when the metal stent expands, it abuts tightly against the blood vessel, avoiding the phenomenon that the outer sheath tube detaches from the blood vessel during surgery, and further facilitating clinicians to operate the guidewire for redirection. When the metal stent contracts, it is out of contact with the blood vessel, allowing clinicians to quickly withdraw the sheath tube from the blood vessel and improving the surgical efficiency.
2. Through the engagement of the ratchet and the pawls, the outer sheath tube can only rotate unidirectionally relative to the inner sheath tube, thereby controlling the metal stent to expand step by step in the blood vessel to adapt to different blood vessel diameters and firmly fix the sheath tube in the blood vessel.

### BRIEF DESCRIPTION OF ACCOMPANY DRAWINGS

FIG. 1 is a schematic structural diagram of an embodiment of a vascular puncture sheath facilitating fixation and redirection according to the invention;

FIG. 2 is a schematic diagram of the expansion of the metal stent in the embodiment of the vascular puncture sheath facilitating fixation and redirection according to the invention;

FIG. 3 is a simplified front view of FIG. 1;

FIG. 4 is a simplified front view of FIG. 2;

FIG. 5 is a cross-sectional view of the structure in FIG. 2;

FIG. 6 is an enlarged view of part A in FIG. 5;

FIG. 7 is an enlarged view of part B in FIG. 5;

FIG. 8 is a bottom view of FIG. 1;

FIG. 9 is a bottom view of FIG. 2;

FIG. 10 is a schematic diagram of the contraction of the metal stent structure of the invention;

FIG. 11 is a schematic diagram of the expansion of the metal stent structure of the invention;

FIG. 12 is a cross-sectional schematic diagram of the adjustment mechanism in FIG. 1 in use;

FIG. 13 is a cross-sectional schematic diagram of the adjustment mechanism in FIG. 2 in use;

FIG. 14 is a cross-sectional view of the structure of the adjustment mechanism in FIG. 13;

FIG. 15 is a simplified structural diagram of the elastic coating of the invention.

FIG. 16 is a structural diagram of a vascular puncture device according to the invention;

FIG. 17 is a structural diagram of a puncture core rod of the invention.

### REFERENCE NUMERALS

1. puncture base, 101. handle part, 102. liquid inlet tube, 2. inner sheath tube, 201. annular convex edge, 3. outer sheath tube, 301. through-hole, 4. metal stent, 401. memory metal wire, 5. chamber, 6. adjustment mechanism, 61. ratchet, 62. pawl, 7. elastic coating, 8. puncture core rod, 81. puncture rod, 82. holding handle.

### SPECIFIC EMBODIMENT OF THE INVENTION

The content of the invention will be described in detail below with reference to the accompanying drawings and embodiments of the specification:

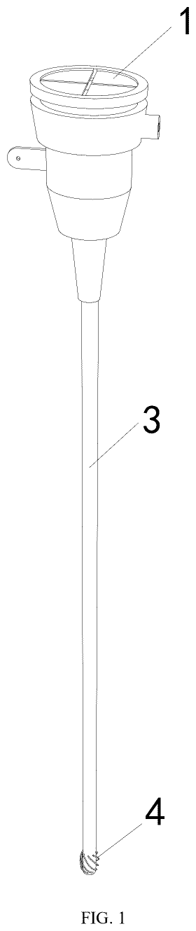

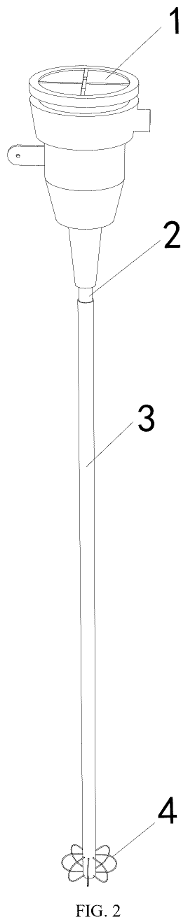

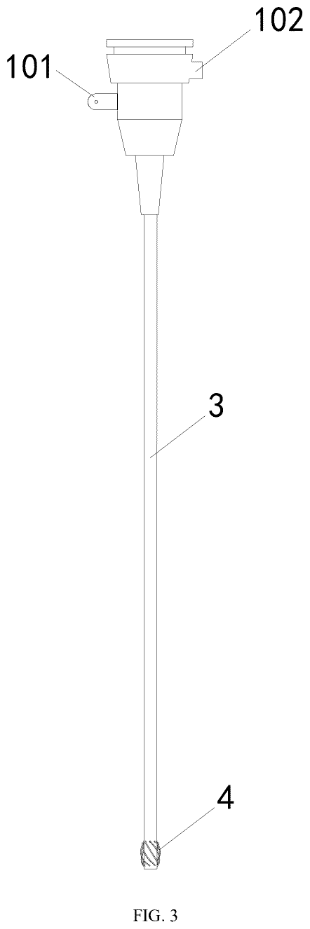

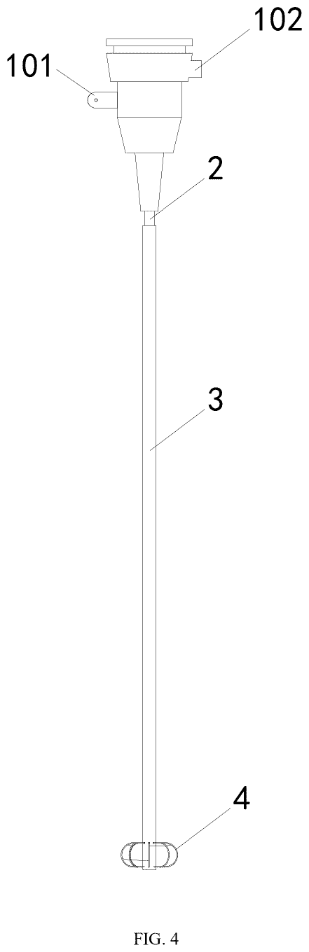

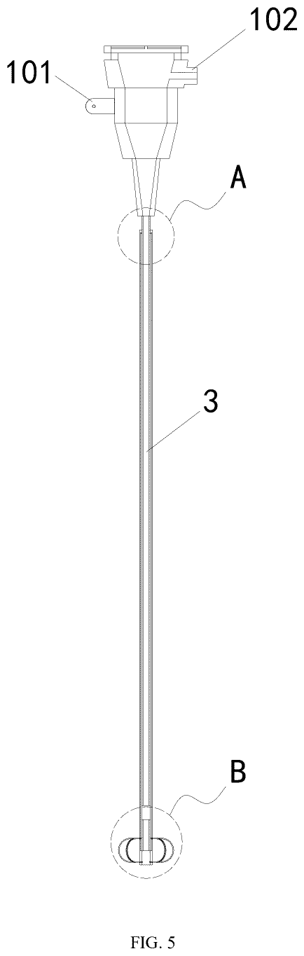

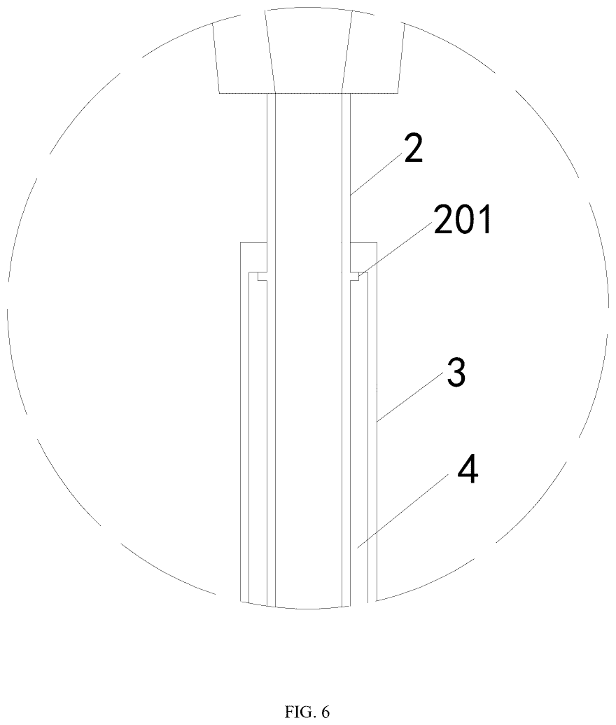

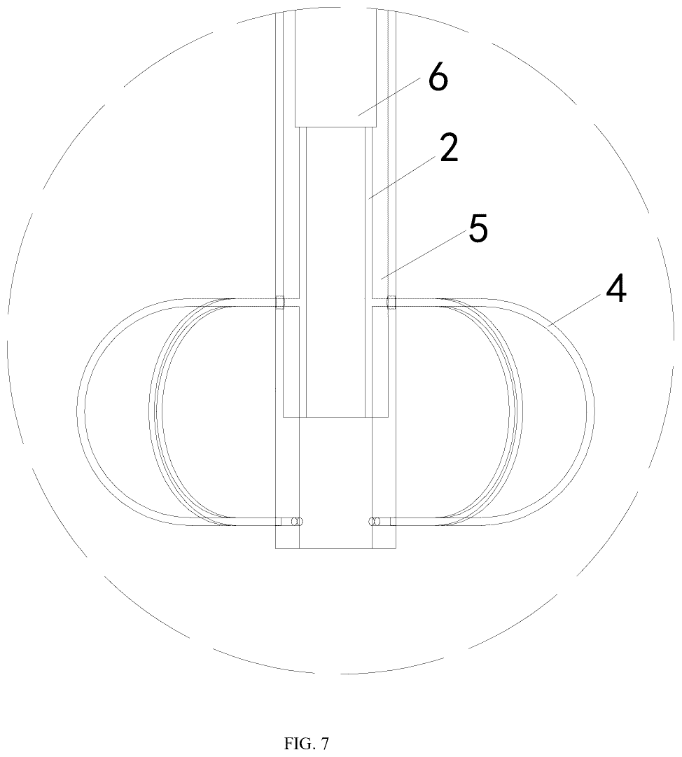

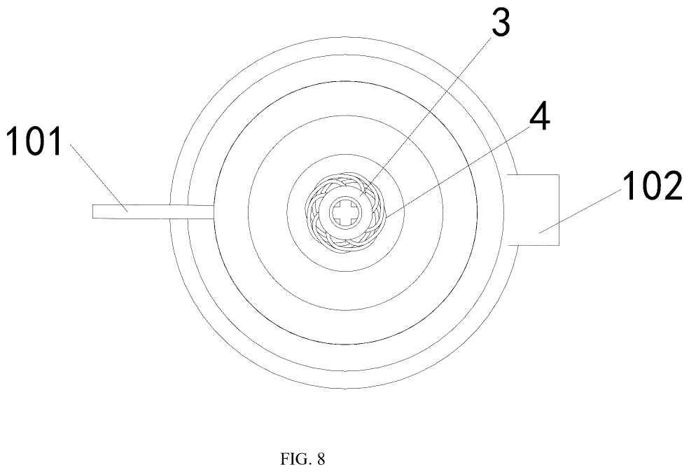

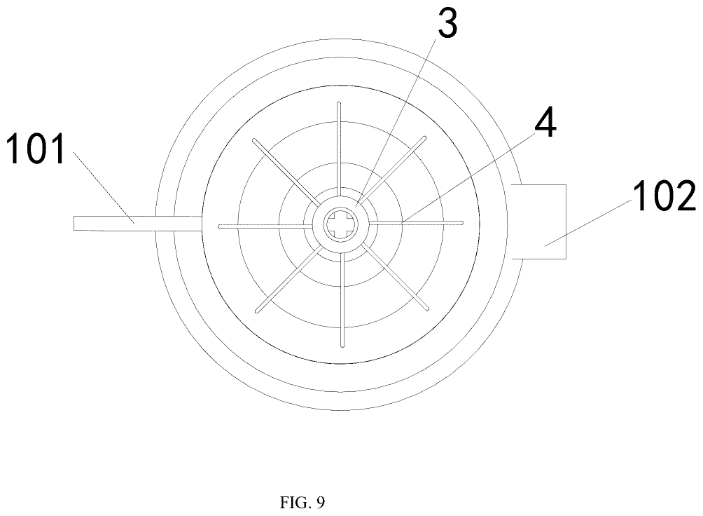

As shown in FIG. 1 to FIG. 14, it is a schematic diagram of an embodiment of a vascular puncture sheath facilitating fixation and redirection provided by the invention:
a vascular puncture sheath facilitating fixation and redirection, comprising:
a puncture base 1;
an inner sheath tube 2, connected to a lower end of the puncture base 1;
an outer sheath tube 3, movably sleeved on an outside of the inner sheath tube 2;
a metal stent 4, fixedly arranged at a lower end of the outer sheath tube 3 and used for fixing the outer sheath tube 3 in the blood vessel.

Wherein, a longitudinally extending chamber 5 is provided between the inner sheath tube 2 and the outer sheath tube 3; a tube wall of the outer sheath tube 3 is provided with a plurality of through-holes 301 communicating with the chamber 5, the metal stent 4 includes a plurality of memory metal wires 401, lower ends of the memory metal wires 401 are fixed on an outer wall of the outer sheath tube 3, and upper ends of the memory metal wires 401 pass through the corresponding through-holes 301 into the chamber 5 and are connected to a tube wall of the inner sheath tube 2;
an adjustment mechanism 6 located above the metal stent 4 is also provided in the chamber 5, the outer sheath tube 3 moves relative to the inner sheath tube 2, thereby driving the adjustment mechanism 6 to control the expansion or contraction of the metal stent 4.

The outer sheath tube 3 can slide up and down and rotate circumferentially relative to the inner sheath tube 2, an outer wall of the inner sheath tube 2 extends outward with an annular convex edge 201 for preventing the outer sheath tube 3 from excessive sliding.

An upper end of the outer wall of the outer sheath tube 3 is provided with a circle of anti-slip lines to increase friction and facilitate the rotation of the outer sheath tube 3.

An upper end surface of the annular convex edge 201 is also provided with an elastic sealing ring to prevent blood from entering the chamber 5.

The adjustment mechanism 6 includes a ratchet 61 arranged around the outer wall of the inner sheath tube 2 and pawls 62 fixedly arranged on an inner wall of the outer sheath tube 3;
the pawls 62 can engage with or disengage from the ratchet 61 as the outer sheath tube 3 slides up and down. When the pawls 62 engage with the ratchet 61, the outer sheath tube 3 can rotate unidirectionally relative to the inner sheath tube 2 to expand the metal stent 4 step by step. When the pawls 62 disengage from the ratchet 61, the outer sheath tube 3 can rotate bidirectionally relative to the inner sheath tube 2 to rotate in the reverse direction and contract the metal stent 4.

The pawls 62 and the outer sheath tube 3 are integrally formed.

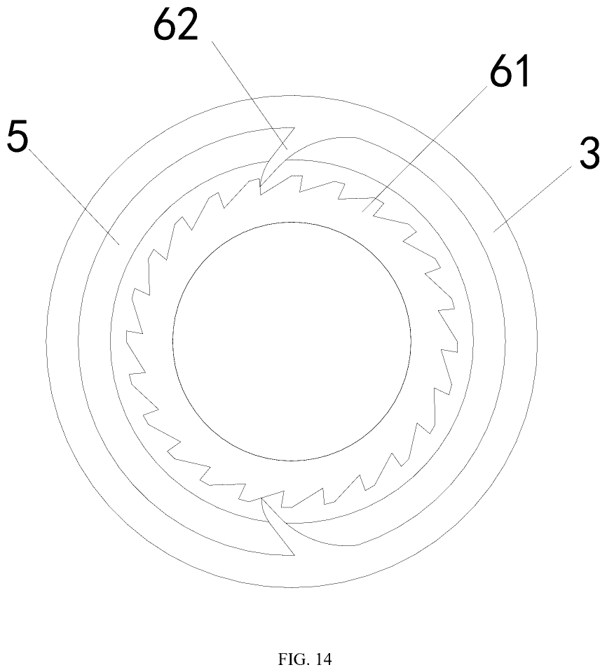

As shown in FIG. 14, there are two pairs of pawls 62, and the two pairs of pawls 62 are respectively arranged on upper and lower sides of the inner wall of the outer sheath tube 3.

The ratchet 61 and the inner sheath tube 2 are integrally formed.

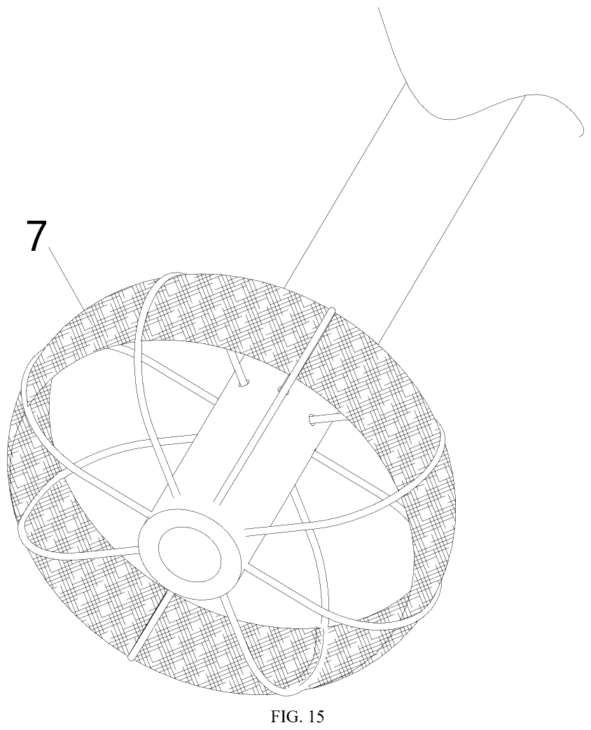

In some embodiments, as shown in FIG. 15, an elastic coating 7 is connected between middle sections of each adjacent memory metal wires 401; the elastic coating 7 can undergo coordinated deformation with the expansion or contraction of the metal stent 4. The distance between upper ends of the memory metal wires 401 and the elastic coating 7 should not be less than the maximum sliding distance of the outer sheath tube 3 relative to the inner sheath tube 2, so that the upper ends of the memory metal wires 401 can smoothly enter the chamber 5 through the through-holes 301.

The through-holes 301 are in clearance fit with the memory metal wires 401, and the space between the through-holes 301 and the memory metal wires 401 is sealed.
a plurality of memory metal wires 401 are arranged at intervals along circumferential direction of the outer wall of the outer sheath tube 3.

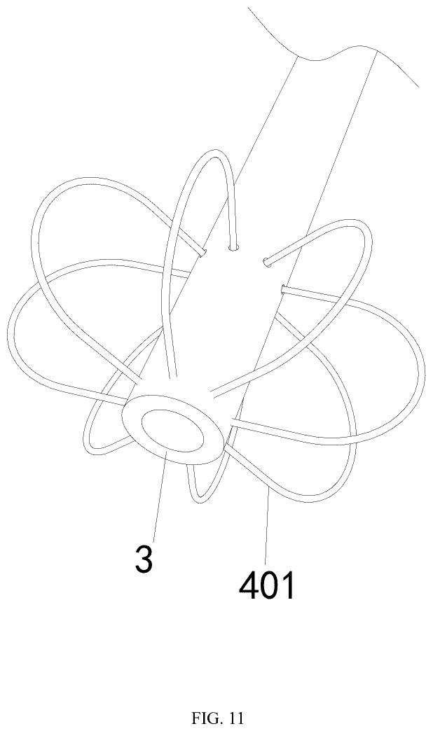

The initial shape of the memory metal wires 401 relative to the outer sheath tube 3, as shown in FIG. 11, is a curved shape with a middle section arching outward. a plurality of memory metal wires 401 are arranged at intervals along the circumferential direction of the outer wall of the outer sheath tube 3 to form a disc-shaped metal stent 4.

Before the outer sheath tube 3 is inserted into the patient's blood vessel, the ratchet 61 and the pawls 62 should be disengaged, and the outer sheath tube 3 should be twisted in the reverse direction relative to the unidirectional rotation direction allowed by the ratchet 61 and the pawls 62, so that the memory metal wires 401 are spirally wound around the outer wall of the outer sheath tube 3. This ensures that when the ratchet 61 and the pawls 62 are engaged and rotate unidirectionally, a plurality of memory metal wires 401 can elastically recover to their initial shape step by step as the outer sheath tube 3 continuously rotates relative to the inner sheath tube 2, thereby realizing the gradual adjustment of the expansion degree of the metal stent 4 according to the blood vessel diameters of different patients.

A left outer wall of the puncture base 1 is provided with a handle part 101 for easy gripping, and a right outer wall of the puncture base 1 is provided with a liquid inlet tube 102 communicating with the puncture base 1.

The liquid inlet tube 102 is connected to an external pipeline and used for delivering medicament (such as heparin and other thrombus-dissolving medicament) into the inner sheath tube 2.

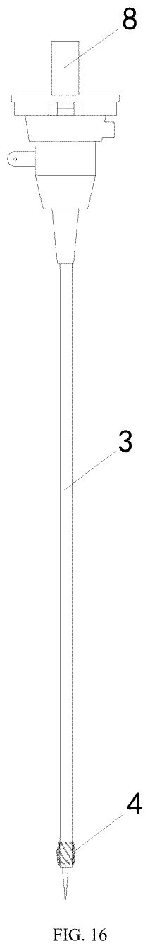

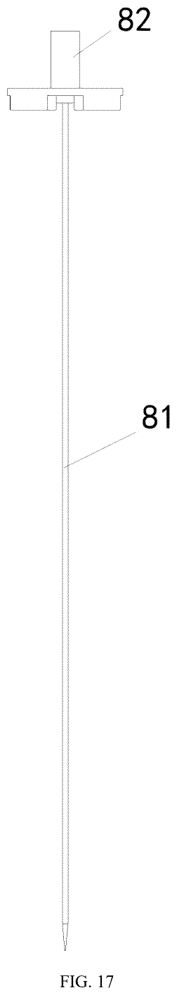

As shown in FIG. 16 to FIG. 17, this is a schematic diagram of an embodiment of a vascular puncture device according to the present invention, which includes the aforementioned vascular puncture sheath facilitating fixation and redirection, and a puncture core rod 8;
the puncture core rod 8 comprises a longitudinally extending puncture rod 81 and a holding handle 82 fixedly arranged at an upper end of the puncture rod 81; the puncture rod 81 is inserted through an upper end of the puncture base 1 and passes out through a lower end of the inner sheath tube 2, a lower end of the puncture rod 81 has a conical structure, and the puncture core rod 8 is provided with a longitudinally extending channel for a guidewire to pass through.

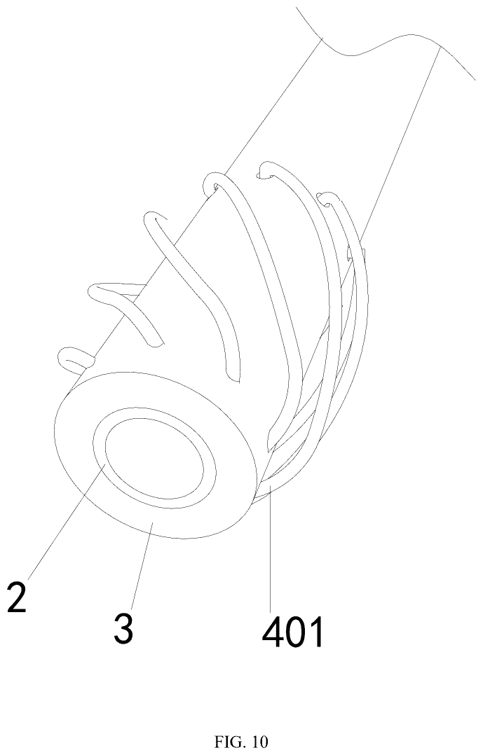

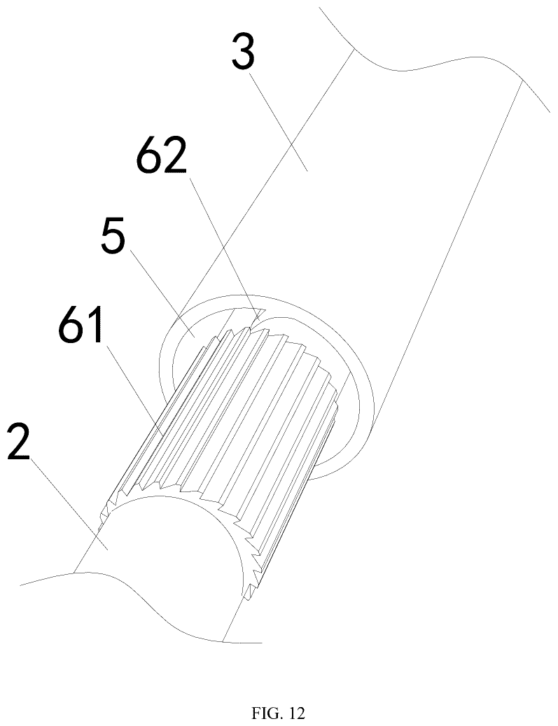

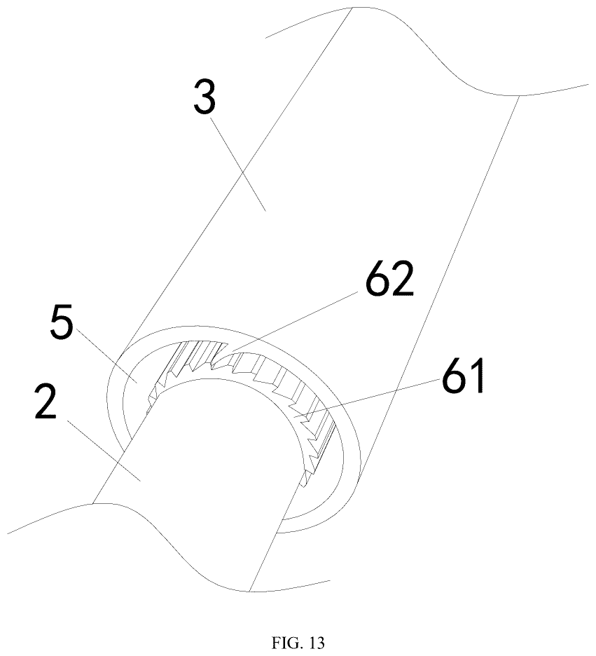

A usage method of the vascular puncture sheath facilitating fixation and redirection in conjunction with the puncture core rod provided by the invention is roughly as follows:
the clinician selects an appropriate puncture point on the patient, and optionally incises the skin at the puncture point according to the actual situation. Then, the puncture sheath with the external puncture core rod 8 and a guidewire inserted is inserted into the blood vessel (at this time, as shown in FIG. 10, the metal stent 4 should be in a contracted state, each memory metal wire 401 is spirally wound around the outer wall of the outer sheath tube 3, and as shown in FIG. 12, the ratchet 61 and the pawls 62 are in a disengaged state). Under the guidance of the guidewire, the puncture core rod and the puncture sheath smoothly enter the patient's blood vessel, and then the puncture core rod 8 is withdrawn. The clinician pushes the outer sheath tube 3 outside the patient's body, so that the outer sheath tube 3 slides downward relative to the inner sheath tube 2 (at this time, as shown in FIG. 13, the ratchet 61 and the pawls 62 are in an engaged state, and the outer sheath tube 3 can only rotate unidirectionally relative to the inner sheath tube 2). Then, by continuously rotating the outer sheath tube 3 in one direction, the memory metal wires 401 spirally wound around the outer wall of the outer sheath tube 3 are elastically recovered step by step, thereby expanding the metal stent 4 and making it tightly abut against the inner wall of the patient's blood vessel, realizing the reliable fixation of the outer sheath tube 3 in the patient's blood vessel, so as to facilitate the clinician to operate the guidewire for redirection in the lumen of the inner sheath tube 2 or operate other surgical instruments for surgery. After the surgery is completed, the clinician pulls the outer sheath tube 3 outside the patient's body, so that the outer sheath tube 3 slides upward relative to the inner sheath tube 2 (at this time, the ratchet 61 and the pawls 62 are disengaged, and the outer sheath tube 3 can rotate freely circumferentially relative to the inner sheath tube 2). Then, the outer sheath tube 3 is rotated in the reverse direction relative to the unidirectional rotation direction of the ratchet 61 and the pawls 62, so that the memory metal wires 401 are spirally wound around the outer wall of the outer sheath tube 3 again. At this time, the puncture sheath is withdrawn from the patient's body or withdrawn until a bottom end of the puncture sheath is located at the puncture port. The metal stent 4 is expanded according to the above operation, so that the metal stent 4 is fixedly supported at the puncture port. The puncture sheath is fixed and left in the blood vessel to avoid secondary puncture of the puncture sheath. Then, the guidewire is inserted and extended toward another end of the blood vessel. After that, the metal stent 4 is retracted, and the puncture sheath is extended along the direction of the guidewire to facilitate the surgery on the other end of the blood vessel.

The above descriptions are only preferred specific implementations of the invention, but the protection scope of the invention is not limited thereto. Any person skilled in the art within the technical scope disclosed by the invention shall replace or change equivalently according to the technical solution of the invention and its inventive concept, and all such replacements or changes shall be covered within the protection scope of the invention.

1. A vascular puncture sheath facilitating fixation and redirection, comprising:
a puncture base;
an inner sheath tube, connected to a lower end of the puncture base;
an outer sheath tube, movably sleeved on an outside of the inner sheath tube;
a metal stent, fixedly arranged at a lower end of the outer sheath tube and used for fixing the outer sheath tube in the blood vessel;
wherein, a longitudinally extending chamber is provided between the inner sheath tube and the outer sheath tube; a tube wall of the outer sheath tube is provided with a plurality of through-holes communicating with the chamber, the metal stent includes a plurality of memory metal wires, lower ends of the memory metal wires are fixed on an outer wall of the outer sheath tube, and upper ends of the memory metal wires pass through the corresponding through-holes into the chamber and are connected to a tube wall of the inner sheath tube;
an adjustment mechanism located above the metal stent is also provided in the chamber, the outer sheath tube moves relative to the inner sheath tube, thereby driving the adjustment mechanism to control the expansion or contraction of the metal stent.

2. The vascular puncture sheath facilitating fixation and redirection according to claim 1, wherein the outer sheath tube can slide up and down and rotate circumferentially relative to the inner sheath tube, an outer wall of the inner sheath tube extends outward with an annular convex edge for preventing the outer sheath tube from excessive sliding.

3. The vascular puncture sheath facilitating fixation and redirection according to claim 2, wherein the adjustment mechanism includes a ratchet arranged around the outer wall of the inner sheath tube and pawls fixedly arranged on an inner wall of the outer sheath tube;
the pawls can engage with or disengage from the ratchet as the outer sheath tube slides up and down; when the pawls engage with the ratchet, the outer sheath tube can rotate unidirectionally relative to the inner sheath tube to expand the metal stent step by step; when the pawls disengage from the ratchet, the outer sheath tube can rotate bidirectionally relative to the inner sheath tube to rotate in the reverse direction and contract the metal stent.

4. The vascular puncture sheath facilitating fixation and redirection according to claim 1, wherein an elastic coating is connected between middle sections of each adjacent memory metal wires.

5. The vascular puncture sheath facilitating fixation and redirection according to claim 1, wherein the through-holes are in clearance fit with the memory metal wires.

6. The vascular puncture sheath facilitating fixation and redirection according to claim 1, wherein a plurality of memory metal wires are arranged at intervals along circumferential direction of the outer wall of the outer sheath tube.

7. The vascular puncture sheath facilitating fixation and redirection according to claim 1, wherein a left outer wall of the puncture base is provided with a handle part for easy gripping, and a right outer wall of the puncture base is provided with a liquid inlet tube communicating with the puncture base.

8. A vascular puncture device, including the vascular puncture sheath facilitating fixation and redirection, and a puncture core rod of claim 1;
the puncture core rod comprises a longitudinally extending puncture rod and a holding handle fixedly arranged at an upper end of the puncture rod; the puncture rod is inserted through an upper end of the puncture base and passes out through a lower end of the inner sheath tube, a lower end of the puncture rod has a conical structure, and the puncture core rod is provided with a longitudinally extending channel for a guidewire to pass through.
